(19) United States
(12) Patent Application Publication
(10) Pub. No.: US 2010/0256630 A1
(43) Pub. Date: Oct. 7, 2010

(54) IRREVERSIBLE ELECTROPORATION (IRE) FOR ESOPHAGEAL DISEASE

(75) Inventors: William C. Hamilton, JR., Queensbury, NY (US); Mark Ortiz, San Jose, CA (US)

Correspondence Address: ANGIODYNAMICS, INC., 14 PLAZA DRIVE, LATHAM, NY, 12110, US

(73) Assignee: ANGIODYNAMICS, INC., Latham, NY (US)

(21) Appl. No.: 12/755,517

(22) Filed: Apr. 7, 2010

Related U.S. Application Data
(60) Provisional application No. 61/167,377, filed on Apr. 7, 2009.

(51) Int. Cl. A61B 18/14

(52) U.S. Cl. 606/41

(57) ABSTRACT

A method for treating Barrett's esophagus and esophageal cancer by using non-thermal electroporation energy to ablate diseased portions of the esophagus which, in effect, prevents stomach acids and other fluids from entering the esophagus thereby alleviating continued deterioration of the esophagus and allows the columnar cells in the lining of the esophagus to assume their normal physical characteristics and functions and.

### CROSS REFERENCE TO RELATED APPLICATIONS

This application claims priority to U.S. Provisional Application Ser. No. 61/167,377 filed Apr. 7, 2009, which is incorporated herein by reference.

### FIELD OF THE INVENTION

The present invention relates to advances in medical procedures aimed at improving the quality and length of life of individuals with Esophageal Disease. More particularly, the present invention relates to a method of using Irreversible Electroporation (IRE) to ablate diseased portions of the esophagus from conditions such as Barrett's Esophagus (BE), squamous cell cancer, adenocarcinoma, sarcoma, cardia cancer, and small cell cancer for improved digestive health.

### BACKGROUND OF THE INVENTION

FIGS. 1 and 2 detail the arrangement of the esophagus (10) with respect to the stomach (12). As shown in FIG. 1, the esophagus (10), or swallowing tube, is a small hose-like tube, which connects the mouth (not shown) to the stomach (12). As the esophagus leaves the mouth, it follows a straight path through the neck (14) and chest (16), passing near the heart (not shown) through a hole (18) in the diaphragm muscle (20) (shown more in detail in FIG. 2), or breathing muscle, and finally entering the stomach (12). FIG. 2 details the structure of the esophagus (10) and stomach (12) wherein the esophagus (10) includes walls composed of muscle that move in wave-like contractions to push food into the stomach (12) and have an inner lining (22), or mucosa, that normally consists of a pinkish-white flat tissue known as squamous epithelium. The inner lining (22) of the esophagus meets the inner lining of the stomach (not shown) at the squamo-columnar junction (26).

Barrett's esophagus is defined as a change in any length of the esophageal epithelium. When the squamous tissue of the esophagus is replaced by red columnar epithelia, the process is known as metaplasia. The metaplastic columnar epithelia may be of two types: gastric or colonic. Barrett's esophagus is a form of colonic metaplasia. In Barrett's esophagus, the columnar tissue (24) of the stomach (12) extends from the junction of the esophagus (10) and stomach (12) upwards into the esophagus (10) towards the mouth (not shown) for a variable distance ranging from a few millimeters to nearly the entire length of the esophagus (10). The metaplasia of Barrett's esophagus may be visible through a gastroscope; however, biopsy specimens of the columnar tissue must be examined under a microscope in order to properly determine if the cells of the tissue are gastric or colonic in nature. Colonic metaplasia is typically identified by the presence of goblet cells in the epithelium and is necessary for a true diagnosis of Barrett's esophagus. Colonic metaplasia is associated with risk of malignancy in genetically susceptible individuals and can potentially lead to the development of esophageal cancer.

The condition of Barrett's esophagus was first described in the 1950's by a British surgeon, Norman Barrett. The exact reasons for development of Barrett's esophagus are unknown. The most widely accepted theory is that a chronic reflux of acid or other stomach contents into the esophagus, known as gastroesophageal reflux disease or GERD, leads to damage to the inner lining (22), or mucosa of the esophagus (10) and causes the inner lining (22), or mucosa to initiate a natural protective/adaptive process/response of healing that results in the presence of columnar epithelia. GERD exists because the lower esophageal sphincter (not shown), a valve located at the junction between the stomach (12) and the esophagus (10) that functions to prevent stomach acids and other contents of the stomach (12) from coming back into the esophagus (10), is weak. As detailed in FIG. 3, weakness of the lower esophageal sphincter (not shown) is due, in part, to the fact that a small portion of the stomach (12) has moved backwards though the opening in the diaphragm (20) and into the chest cavity (16) creating the presence of a hernia, called a hiatal hernia (28); wherein the upper few centimeters of the stomach (12) slide back and forth between the abdomen interfering with the function of the lower esophageal sphincter.

As discussed earlier, and shown in detail in FIG. 4, in Barrett's esophagus, the columnar tissue (24) of the stomach (12) extends from the junction of the esophagus (10) and stomach (12), as at the squamo-columnar junction (26), upwards into the esophagus (10). Chronic or severe Barrett's esophagus is developed over years, and although it is believed that 10 to 20 million people in the U.S. have acid reflux, only 1 out of 10 people with severe acid reflux problems actually have Barrett's esophagus. Those individuals with Barrett's esophagus have a 30 to 40 percent increased risk of developing esophageal cancer.

Esophageal cancer is the result of uncontrolled cell growth in the esophagus. Esophageal cancer is divided into two major types—squamous cell carcinoma (30) and adenocarcinoma (32). FIG. 5 details that squamous cell carcinomas (30) develop in the squamous cells that line the esophagus (10). These cancers normally occur in the upper to middle part of the esophagus (10). Adenocarcinomas (32) typically develop in the glandular tissue in the lower portion of the esophagus (10) in the region where the esophagus (10) and the stomach (12) join. Although esophageal cancer is not as common as breast, lung, prostate or colon cancers, esophageal cancer is; however, rapidly increasing in frequency, faster than any other type of cancer.

Although treatments for Barrett's esophagus are available and readily practiced, there is no reliable way of determining which patients with Barrett's esophagus will go on to develop esophageal cancer. Current treatments for Barrett's esophagus include routine endoscopy and biopsy every 12 months or so while the underlying reflux are controlled with non-steroidal anti-inflammatory drugs (NSAIDS), like aspirin, or with proton pump inhibitor (PPI) drugs in combination with other measures to prevent reflux. Endoscopy and biopsy are processes that involve surveillance of the esophagus to detect changes in the lining of the esophagus. If these changes exist, a patient is at higher risk of having Barrett's esophagus progress to cancer. For treatment in more extreme or advanced cases of Barrett's esophagus or esophageal cancer, procedures include radiation therapy, systemic chemotherapy, photodynamic therapy (PDT) and laser treatment. Other well known procedures are Endoscopic mucosal resection (EMR) or esophagectomy and fundoplication (anti-reflux) surgeries. Some physicians are experimentally trying to destroy the Barrett's lining with the hope that normal squamous cells will grow back. These experimental procedures include argon plasma coagulation (APC) and multipolar electro-coagulation (MPEC).

The type of treatment is selected depending upon a number of factors including the grade of cell change in the lining of the esophagus, size and location of the cell change, and the patient's health. Many of the treatments are associated with a variety of side effects including but not limited to pain and tenderness at the procedure site, fluid developing in the lungs, dry/sore mouth and throat, difficulty swallowing swelling of the mouth and gums, fatigue, nausea, vomiting, diarrhea, hair loss and skin changes. Specifically, radiation therapy, systemic chemotherapy, photodynamic therapy (PDT) and laser treatment are associated, as well, with a fair amount of surgically related setbacks including complications such as large and difficult to manipulate operating mechanisms and the inability to control therapy to the affected area. These techniques, historically, are non-selective in that cell death is mediated by extreme heat or cold temperatures. These methods also adversely affect blood vessels, nerves, and connective structures adjacent to the ablation zone. Disruption of the nerves locally impedes the body's natural ability to sense and regulate homeostatic and repair processes at and surrounding the treated region. Disruption of the blood vessels prevents removal of debris and detritus. This also prevents or impedes repair systems, prevents homing of immune system components, and generally prevents normal blood flow that could carry substances such as hormones to the area. Without the advantage of a steady introduction of new materials or natural substances to a damaged area, reconstruction of the blood vessels and internal linings become retarded as redeployment of cellular materials is inefficient or even impossible. Therefore, historical extreme temperature treatments do not leave tissue in an optimal state for self-repair in regenerating the region.

Improvements in medical techniques have rekindled interest in the surgical treatment of Barrett's esophagus and esophageal cancer, wherein much of the associated risks, side effects and complications of conventional techniques are overcome. These recent developments offer an opportunity to advance the regenerative process following treatment. Irreversible Electroporation or (IRE) is one such technique that is pioneering the surgical field with improved treatment of tissue ablation. IRE has the distinct advantage of non-thermally inducing cell necrosis without raising/lowering the temperature of the area being treated, which avoids some of the adverse consequences associated with temperature changes of ablative techniques such as radiation therapy, systemic chemotherapy, photodynamic therapy (PDT) and other earlier forms of laser treatment. IRE also offers the ability to have a focal and more localized treatment of an affected area. The ability to have a focal and more localized treatment is beneficial when treating the delicate intricacies of organs such as the esophagus.

IRE is a minimally invasive ablation technique in which permeabilization of the cell membrane is effected by application of micro-second, milli-second and even nano-second electric pulses to undesirable tissue to produce cell necrosis only in the targeted tissue, without destroying critical structures such as airways, ducts, blood vessels and nerves. More precisely, IRE treatment acts by creating defects in the cell membrane that are nanoscale in size and that lead to a disruption of homeostasis while sparing connective and scaffolding structure and tissue. Thus, destruction of undesirable tissue is accomplished in a controlled and localized region while surrounding healthy tissue, organs, etc. is spared. This is different from other thermal ablation modalities known for totally destroying the cells and other important surrounding organs and bodily structures.

### BRIEF SUMMARY OF THE DISCLOSURE

The present invention relates to methods for treating tissue, more particularly to treating diseased tissue of the esophagus, through utilization of Irreversible Electroporation (IRE) to non-thermally ablate diseased tissue and enhance digestive functions in patients with Barrett's esophagus and esophageal cancer.

It is a purpose of this invention to successfully treat target regions of diseased tissue of the esophagus affected by Barrett's esophagus and esophageal cancer through IRE ablation. IRE involves the application of energy sources capable of generating a voltage configured to successfully ablate tissue through the utilization of perfusion electrode balloons, flexible devices, probes such as monopolar, bipolar, or multiple probes (i.e. combinations of monopolar or bipolar probes arranged in a variety of configurations, monopolar and bipolar probes used together, or a series of separate or mixed groups of monopolar or bipolar probes), electrode arrays, and other devices available in electro-medicine. IRE ablation devices are available in various combinations and configurations in order to accommodate the ablation of multiple shapes, sizes and intricate portions of the diseased tissue. Examples of IRE devices applicable to this invention are described in U.S. patent application Ser. No. 12/413,332 filed Mar. 27, 2009 and U.S. Ser. No. 61/051,832 filed May 15, 2008, both of which are incorporated herein.

The present invention involves the method of treating Barrett's esophagus and esophageal cancer using IRE typically through endotracheal procedures including the steps of obtaining access to the diseased area by positioning one or more energy delivery devices coupled to an IRE device within a target region of diseased tissue; applying IRE energy the target region to ablate the tissue; disconnecting the energy source from the IRE probe and withdrawing the probe. More specifically, the invention involves ablating diseased tissue of esophagus. Although the endotracheal method is preferred, it is conceivable that other methods such as open surgical, percutaneous or perhaps laparoscopic procedures may be used to carry out IRE treatment. Specifics involving the method of the present invention is directed towards treatment of a diseased esophagus, the method; however, can also be used to treat other organs or areas of tissue to include, but not limited to areas of the digestive, skeletal, muscular, nervous, endocrine, circulatory, reproductive, lymphatic, urinary, or other soft tissue or organs; and more particularly, areas of the lung, liver, prostate, kidney, pancreas, colon, urethra, uterus and brain, among others.

### BRIEF DESCRIPTION OF THE DRAWINGS

FIG. 1 is a perspective view of a normal esophagus and stomach.

FIG. 2 is a perspective internal view of a normal esophagus and stomach.

FIG. 3 is a perspective view of a hiatal hernia.

FIG. 4 is a perspective internal view of a Barrett's esophagus.

FIG. 5 is a perspective view depicting the typical locations of squamous cell cancer and adenocarcinoma.

FIGS. 6A and 6B are perspective views of the endotracheal procedure for performing IRE on an esophagus affected by Barrett's esophagus.

FIG. 7 is a perspective view of the endotracheal procedure for performing IRE on an esophagus affected by esophageal cancer.

FIG. 8 is a flowchart showing the method of treating patients with Barrett's esophagus and esophageal cancer using IRE ablation.

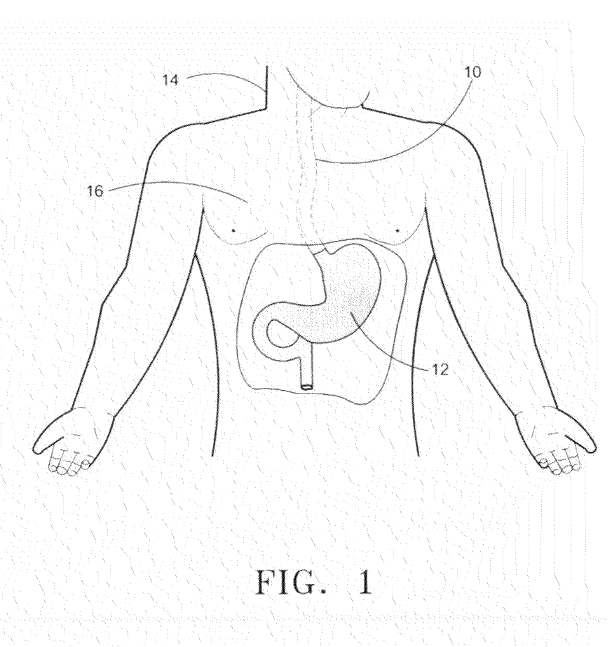

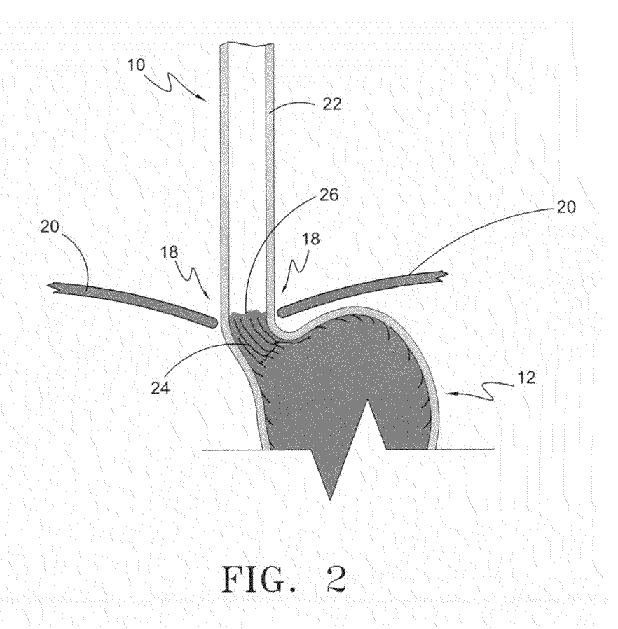

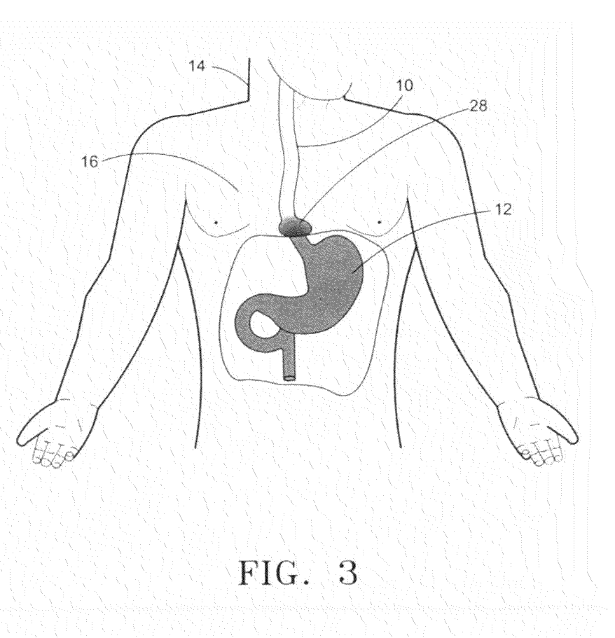

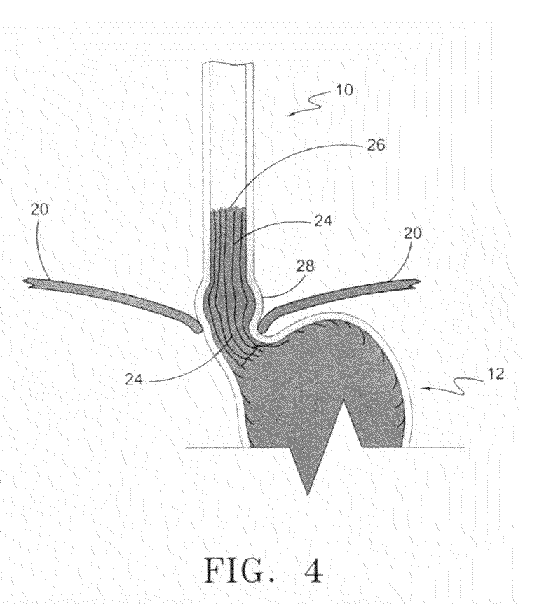

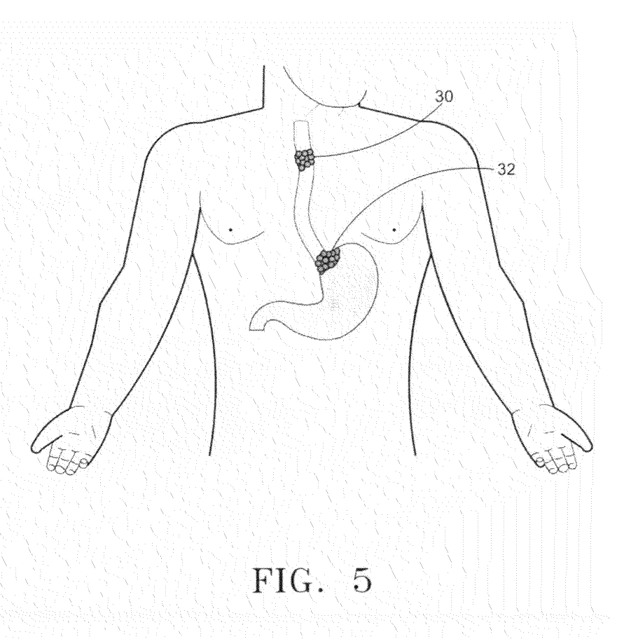

### DETAILED DESCRIPTION OF THE INVENTION

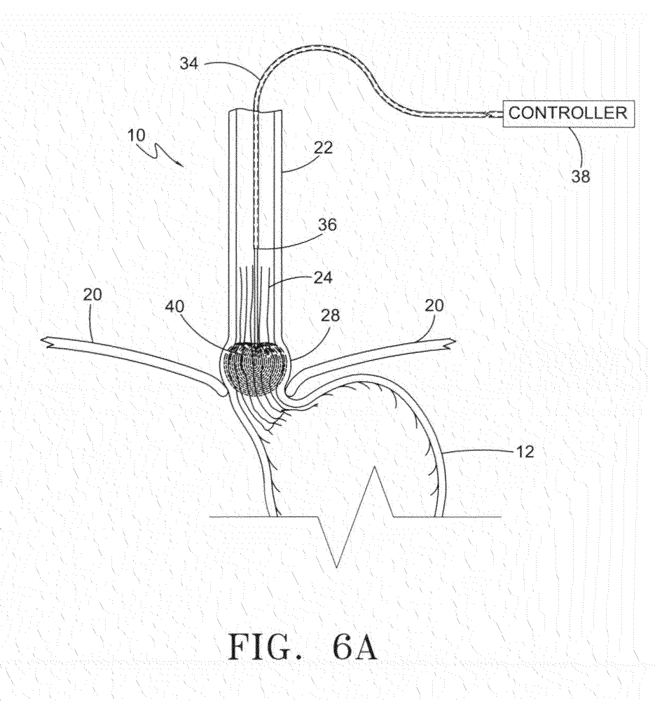

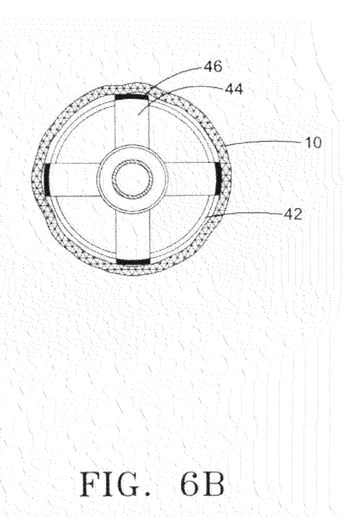

FIGS. 6A and 6B show the endotracheal method of performing IRE on an esophagus (10) affected by Barrett's esophagus. A catheter (34) is advanced through the trachea (not shown) down into the esophagus (10) to a diseased region, which in the case of Barrett's esophagus is the columnar lining (24) of the esophagus (10). Advancement through the trachea (not shown) is relatively simple and may optionally require a guidewire to select the advancement route through to the esophagus (10). Steering of the catheter (34) may be effected under real time imaging using Video Assisted Thoracic Surgery (VATS). Once the catheter (34) is in place inside the diseased region (24), a flexible IRE device (36) is inserted through the catheter (34) to the diseased region (24) of the esophagus (10). The flexible IRE device (36) is used in the endotracheal method because it allows for the device to be easily steered through and properly positioned within the esophagus (10). FIG. 6B shows that the IRE device may be an electrode balloon. For purposes of allowing air flow through the trachea during the procedure, perfusion balloons are often times employed. Perfusion balloons do not obstruct the flow of air through the trachea therefore allowing the procedure to be carried out without time restrictions. Although FIG. 6B depicts and electrode balloon (42), the method is not limited to such, as other devices may also be employed to effectively carry out the procedure. An example of an IRE electrode balloon (42) applicable to this invention, as mentioned above, is detailed in U.S. application Ser. No. 12/413,332 filed Mar. 27, 2009 which is incorporated herein by reference. In the instant application, the IRE electrode balloon (42) is carefully designed so as to encourage air flow during the procedure. The IRE electrode balloon (42) includes legs (44) with electrodes (46). When the IRE electrode balloon (42) is positioned within the diseased region (24) of the esophagus (10), the electrodes (46) come into contact with the inner lining (22) of the esophagus. An IRE power source (38) is powered on and IRE energy (40) is applied to ablate the tissue of the diseased region (24). After application of the desired amount of IRE energy (40), the IRE power source (38) is powered down and the flexible IRE device (36) is removed. To treat large diseased regions (24), the IRE device (36) may be retracted back into the catheter (34), moved and redeployed in an adjacent diseased region (24) of the esophagus (10).

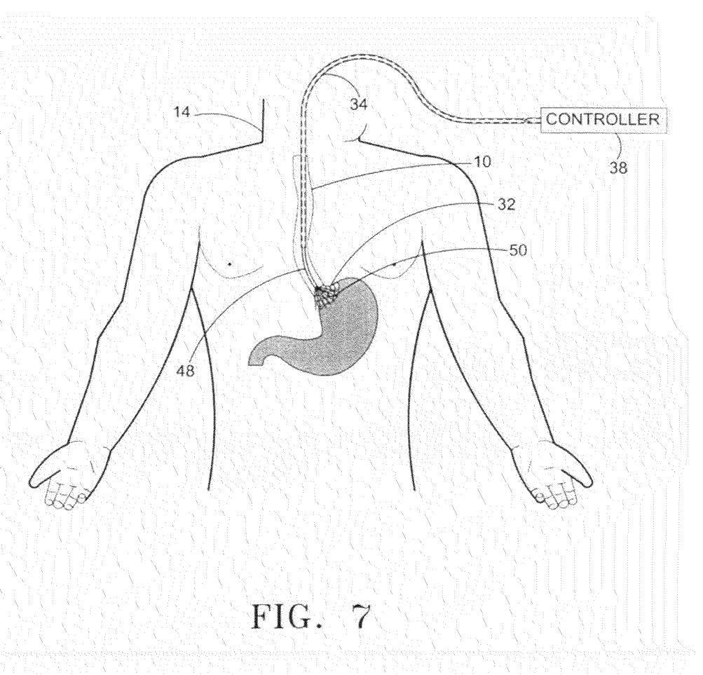

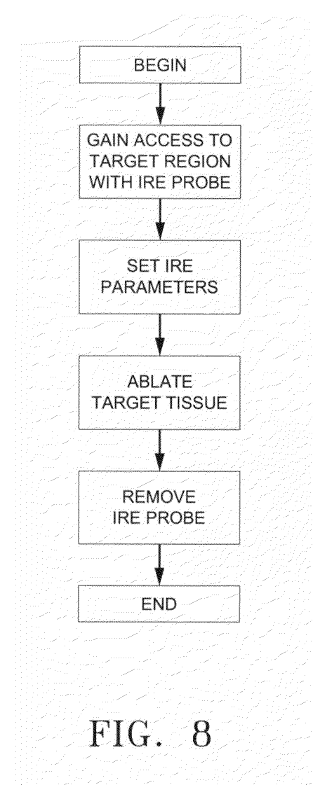

FIG. 7 shows the endotracheal method of performing IRE on an esophagus (10) affected by esophageal cancer. A catheter (34) is advanced through the trachea (not shown) down into the esophagus (10) to a diseased region, which in the case of esophageal cancer, is the adenocarcinoma (32). Advancement through the trachea (not shown) is relatively simple and will optionally require a guidewire to select the advancement route through to the esophagus (10). Steering of the catheter (34) is effected under real time imaging using video assisted thoracic surgery (VATS). Once the catheter (34) is in place inside the diseased region (32), a flexible IRE device (48) is inserted through the catheter (34) to the diseased region (32) of the esophagus (10). The flexible IRE device (48) is used in the endotracheal method because it allows for the device to be easily steered through and properly positioned within the esophagus (10). Typically this device is an IRE probe (48); however, the method is not limited to such and may include other devices. With the flexible IRE device (48) within the diseased region (32) of the esophagus (10), an IRE power source (38) is powered on and IRE energy (50) is applied to ablate the tissue of the diseased region (32). After application of the desired amount of IRE energy (50), the IRE power source (38) is powered down and the flexible IRE device (48) is removed. To treat large diseased regions (32), the IRE device (48) may be retracted back into the catheter (34), moved and redeployed in an adjacent diseased region (32) for treatment.

Ablation of the targeted region of diseased tissue (24) or (32) is achieved with an IRE generator as the power source, utilizing a standard wall outlet of 110 volts (v) or 230v with a manually adjustable power supply depending on voltage. The generator should have a voltage range of 100v to 10,000v and be capable of being adjusted at 100v intervals. The applied ablation pulses are typically between 20 and 100 microseconds in length, and capable of being adjusted at 10 microsecond intervals. The preferred generator should also be programmable and capable of operating between 2 and 50 amps, with test ranges involving an even lower maximum where appropriate. It is further desired that the IRE generator includes 2 to 6 positive and negative connectors, though it is understood that the invention is not restricted to this number of connectors and may pertain to additional connector combinations and amounts understood in the art and necessary for optimal configurations for effective ablation. Preferably, IRE ablation involves 90 pulses with a maximum field strength of 400V/cm to 3000V/cm between electrodes. Pulses are applied in groups or pulse-trains where a group of 1 to 15 pulses are applied in succession followed by a gap of 0.5 to 10 seconds. Although pulses can be delivered using probes, needles, and electrodes each of varying lengths suitable for use with percutaneous, laparoscopic and open surgical procedures; due to the delicate intricacies and general make-up of the esophagus, it is preferable that a flexible device be used to ensure proper placement and reduced risk of perforation, abrasion, or other trauma to the esophagus.

Although preferred specifics of IRE ablation devices are set forth above, electro-medicine provides for ablation processes that can be performed with a wide range of variations. For instance, some ablation scenarios can involve 8 pulses with a maximum field strength between electrodes of 250V/cm to 500V/cm, while others require generators having a voltage range of 100kV-300kV operating with nano-second pulses with a maximum field strength of 2,000V/cm to, and in excess of, 20,000V/cm between electrodes. Electrodes can be made using a variety of materials, sizes, and shapes known in the art, and may be spaced at an array of distances from one another. Conventionally, electrodes have parallel tines and are square, oval, rectangular, circular or irregular shaped; having a distance of 0.5 to 10 centimeters (cm) between two electrodes; and a surface area of 0.1 to 5 cm2.

FIG. 7 is a flowchart detailing the basic method of performing IRE ablation on patients with Barrett's esophagus an esophageal cancer. As detailed above, access to the diseased region is typically gained endotracheally. Once the IRE device is connected and in proper position, the IRE parameters are set. These parameters may vary and are selected depending upon several factors such as the diseased state, patient health and anatomy, and other considerations. After establishing and setting the required IRE energy parameters, the diseased region of the esophagus is ablated and the IRE device is removed. Thus, focal tissue ablation of the esophagus is achieved without causing harm to surrounding tissue and/or organs.

IRE treatment of both diseased conditions, Barrett's esophagus and esophageal cancer, necrosis the bad or columnar/squamos cells which thereafter are slowly removed from the body through natural processes, and the good or normal cells are allowed to regenerate. This type of non-thermal treatment does not affect or destroy elastins or surrounding connective tissue thereby sparing and preserving the natural structure, and restoring the functions of the esophagus.

An unlimited number of variations and configurations for the present invention could be realized, The foregoing discussion describes merely exemplary embodiments illustrating the principles of the present invention, the scope of which is recited in the following claims. Those skilled in the art will readily recognize from the description, the claims, and drawings that numerous changes and modifications can be made without departing from the spirit and scope of the invention. Accordingly, the scope of the invention is not limited to the foregoing specification.

What is claimed is:

1. A method of treating an esophagus including the steps of:
a. obtaining access to the esophagus, wherein the esophagus contains a diseased region;
b. positioning at least one energy delivery device within the diseased region, wherein the energy delivery device is coupled to an electroporation energy source; and
c. applying electroporation energy to non-thermally ablate a portion of the diseased region.

2. The method of claim 1, wherein the step of obtaining access further comprises obtaining access endotracheally.

3. The method of claim 1, wherein the step of applying electroporation energy further comprises the application of energy from at least one energy delivery device selected from the group consisting of at least one of electrode balloons, monopolar probes, bipolar probes, multipolar probes, electrode arrays, and any combination thereof.

4. The method of claim 1, wherein the step of applying electroporation energy further comprises the application of energy using an electrode balloon.

5. The method of claim 1, wherein the step of applying electroporation energy further comprises the application of energy using a perfusing electrode balloon.

6. The method of claim 1, wherein the step of applying electroporation energy further comprises the application of energy using an IRE probe.

7. The method of claim 1, wherein the step of applying electroporation energy further comprises the application of energy using a catheter and IRE probe.

8. The method of claim 1, wherein the step of applying electroporation energy further comprises:
inserting the at least one energy delivery device into the catheter prior to ablation;
retracting the at least one energy delivery device within the catheter after ablation;
moving the catheter;
redeploying the at least one energy delivery device in an adjacent diseased region; and
ablating the adjacent diseased region.

9. The method of claim 5, wherein the step of ablating further comprises ablation of the diseased region that is caused by Barrett's esophagus.

10. The method of claim 7, wherein the step of ablating further comprises ablation of he diseased region that is caused by esophageal cancer.

11. The method of claim 5, further comprising placing the electrode balloon in contact with the inner lining of the esophagus.

12. The method of claim 8, wherein the step of ablating further comprises ablation using an energy field strength in the range of 100V/cm to greater than 10,000V/cm.

13. The method of claim 8, wherein the step of ablating further comprises ablating the diseased region until bad cells necrose while allowing good cells to regenerate.

14. The method of claim 13, wherein the step of ablating further comprises sparing the esophagus from destruction and preserving connective tissue surrounding the esophagus.

15. The method of claim 1, wherein the step of applying electroporation energy further comprises applying energy using a flexible device.

16. The method of claim 8, wherein the step of applying electroporation energy further comprises applying energy using a flexible device.
